(19) United States
(12) Patent Application Publication
(10) Pub. No.: US 2009/0192063 A1
(43) Pub. Date: Jul. 30, 2009

(54) Final Drive and Powershift Transmission Lubricants

(75) Inventors: David B. Gray, Darmstadt (DE); Charles A. Passut, Midlothian, VA (US); Lee D. Saathoff, Glen Allen, VA (US)

Correspondence Address: AFTON CHEMICAL CORPORATION;LUEDEKA, NEELY & GRAHAM, PC, P.O. BOX 1871, KNOXVILLE, TN, 37901, US

(73) Assignee: Afton Chemical Corporation, Richmond, VA (US)

(21) Appl. No.: 12/337,036

(22) Filed: Dec. 17, 2008

Related U.S. Application Data
(60) Provisional application No. 61/023,460, filed on Jan. 25, 2008.

(51) Int. Cl. C10M 141/10; C10M 135/10; G01N 33/30; C10M 139/06

(52) U.S. Cl. 508/372; 508/403; 508/382; 73/10; 73/53.05

(57) ABSTRACT

There is disclosed a lubricating composition comprising an oil of lubricating viscosity formulated with an additive package comprising at least one overbased metal detergent, at least one neutral metal detergent, and at least one phosphorus-based wear preventative. Methods for improving oxidation performance in a TO-4 fluid for use in heavy vehicle machinery are also disclosed.

### RELATED APPLICATIONS

The present application claims the benefit of priority to U.S. Provisional Application No. 61/023,460 filed on Jan. 25, 2008.

### FIELD

The present disclosure relates to lubricating compositions, additive packages, and methods for lubricating both a transmission and a diesel engine. Diesel engine lubricants must pass a series of tests. Similarly, transmission lubricants are required to pass another series of tests. Surprisingly, the inventive composition may be used to create lubricants for both engine and transmission use.

### BACKGROUND

Modern heavy vehicle machinery, for example earth moving equipment, is continually updated to meet ever increasing consumer demands. Significant improvements in transmissions and final drives in heavy vehicle machinery have increased equipment durability and productivity and new and diverse friction materials are continually being developed to further enhance performance. Providing the correct lubricants to support these new designs plays a significant role in achieving maximum life and performance for the vehicle.

In the early 1990s, Caterpillar Corporation introduced a new set of transmission and drive train fluid requirements, designated as “Caterpillar TO-4” the Jun. 23, 2005 specification of which is incorporated herein by reference, for use in Caterpillar's heavy vehicle machinery. All TO-4 oils must comply with a number of fluid properties including certain wear, viscometric and friction conditions as set out in the TO-4 specification. Many of the additives used in final drive and powershift transmission (FDPT) lubricants are multifunctional and there is often a conflict generated between properties. These conflicts inevitably mean that additives must be carefully selected and balanced. Accordingly, it has proven difficult for additive companies to meet TO-4 requirements, much less improve significantly on any of the performance thresholds. Surprisingly, the fluids of the present invention are able to meet the TO-4 specification and significantly improve upon the FZG scuffing rating. In addition, the present invention is able to accomplish this improvement with a low treat rate additive package which reduces additive shipping costs, improves plant through-put, and provides economic benefits to lubricant blenders in terms of lower net additive treat costs.

### SUMMARY

In accordance with the disclosure, the present invention relates to CAT TO-4 compliant fluid compositions comprising a base oil, at least one metal phenate, at least one metal dialkylnapthalene sulfonate and at least one phosphorus-based wear preventative.

Other embodiments of the invention include the TO-4 fluid composition mentioned above, wherein the at least one metal phenate is selected from the group consisting of calcium phenate, magnesium phenate, zinc phenate, and mixtures thereof. In other embodiments the metal phenate is calcium phenate.

Other embodiments of the invention include the TO-4 fluid composition mentioned above, wherein the metal dialkylnapthalene sulfonate is selected from the group consisting of calcium dialkylnapthalene sulfonate, magnesium dialkylnapthalene sulfonate, zinc dialkylnapthalene sulfonate, and mixtures thereof. In other embodiments the metal dialkylnapthalene sulfonate comprises a zinc dinonylnapthalene sulfonate.

Other embodiments of the invention include TO-4 fluid compositions of the present invention wherein the at least one phosphorus-based wear preventative comprises at least one metal dihydrocarbyl dithiophosphate compound. In other embodiments the at least one metal dihydrocarbyl dithiophosphate compound comprises at least one zinc dihydrocarbyl dithiophosphate compound.

Other embodiments of the invention include TO-4 fluids of the present invention wherein the composition contains from about 200 to about 800 ppm phosphorus from the metal dihydrocarbyl dithiophosphate compound. Other embodiments of the invention include TO-4 fluids wherein said composition contains from about 200 to 400 ppm phosphorus from the metal dihydrocarbyl dithiophosphate compound.

Other embodiments of the invention include any one of the lubricant compositions mentioned above wherein the lubricating composition is TO-4 compliant having an FZG scuffing load stage score of 12.

In another embodiment of the invention an additive package is disclosed comprising at least one metal phenate, at least one metal dialkylnapthalene sulfonate and at least one phosphorus-based wear preventative

Other embodiments of the invention include the additive package mentioned above wherein the at least one metal phenate is selected from the group consisting of calcium phenate, magnesium phenate, zinc phenate, and mixtures thereof.

Other embodiments of the invention include the additive package mentioned above wherein the metal phenate is calcium phenate.

Other embodiments of the invention include the additive package mentioned above wherein the metal dialkylnapthalene sulfonate is selected from the group consisting of calcium dialkylnapthalene sulfonate, magnesium dialkylnapthalene sulfonate, zinc dialkylnapthalene sulfonate, and mixtures thereof.

Other embodiments of the invention include the additive package mentioned above wherein the metal dialkylnapthalene sulfonate comprises a zinc dinonylnapthalene sulfonate.

Other embodiments of the invention include the additive package mentioned above wherein the at least one phosphorus-based wear preventative comprises at least one metal dihydrocarbyl dithiophosphate compound. In another embodiment the at least one metal dihydrocarbyl dithiophosphate compound comprises at least one zinc dihydrocarbyl dithiophosphate compound.

Other embodiments of the invention include the additive package mentioned above wherein the additive package contains from about 200 to about 800 ppm phosphorus from the metal dihydrocarbyl dithiophosphate compound.

Other embodiments of the invention include the additive package mentioned above wherein the additive package contains from about 200 to 400 ppm phosphorus from the metal dihydrocarbyl dithiophosphate compound.

Another embodiment of the invention includes a method for improving the FZG scuffing protection of a vehicle engine or transmission comprising the steps of: (1) adding to the vehicle engine or transmission any one of the fluid compositions provided above; and (2) operating the vehicle.

Another embodiment of the invention includes a method of lubricating a machine part comprising lubricating the machine part with any one of the lubricant compositions mentioned above. The machine part may comprise a gear, an axle, a differential, an engine, a crankshaft, a transmission, or a clutch, and wherein the transmission is selected from the group consisting of an automatic transmission, a manual transmission, an automated manual transmission, a semi-automatic transmission, a dual clutch transmission, a continuously variable transmission, and a toroidal transmission. In some embodiments the transmission comprises a continuously slipping torque converter clutch, a slipping torque converter, a lock-up torque converter, a starting clutch, one or more shifting clutches, or an electronically controlled converter clutch. In other embodiments the gear is selected from the group consisting of an automotive gear, a stationary gearbox, and an axle. In other embodiments the gear is selected from the group consisting of a hypoid gear, a spur gear, a helical gear, a bevel gear, a worm gear, a rack and pinion gear, a planetary gear set, and an involute gear. In other embodiments the differential is selected from the group consisting of a straight differential, a turning differential, a limited slip differential, a clutch-type limited slip differential, and a locking differential. In other embodiments the engine is selected from the group consisting of an internal combustion engine, a diesel engine, a rotary engine, a gas turbine engine, a four-stroke engine, and a two-stroke engine. In other embodiments the engine comprises a piston, a bearing, a crankshaft, and/or a camshaft.

Another embodiment of the invention includes a method for testing the lubricant properties of a composition using a testing apparatus comprising lubricating said testing apparatus with a any one of the lubricant compositions disclosed above, the testing apparatus selected from the group consisting of: a Brookfield viscometer, any Vickers Test apparatus, an SAE No. 2 friction test machine, an electric motor-driven Hydra-Matic 4L60-E automatic transmission, ASTM D 471 or D 676 Elastomer Compatibility test equipment, NOACK volatility procedure machine, any test apparatus necessary for ASTM D 2882, D 5182, D 4172, D3233, and D2782 Wear Procedures, ASTM Foaming Procedure apparatus, test apparatus necessary for ASTM D 130 Copper Corrosion test, test equipment specified by the International Harvester Procedure Method BT-9 Rust Control test, test apparatus required by ASTM D 892 Foaming test, test apparatus required by ASTM D 4998 Gear Anti-Wear Performance test, Link M1158 Oil/Friction Machine, L-33-1 Test Apparatus, L-37 Test Apparatus, L-42 Test Apparatus, L-60-1 Test Apparatus, Strama 4-Square Electric Motor-Driven Procedure Machine, FZG Test Apparatus and parts, SSP-180 Procedure Machine, test apparatus for ASTM D 5579 High Temperature Cyclic Durability Procedure, Sauer-Danfoss Series 22 or Series 90 Axial Piston Pump, John Deere Synchro-Plus transmission, an SRV-friction wear tester, a 4-ball test apparatus, an LFW-1 test apparatus, a sprag clutch over-running wear test (SCOWT) apparatus, API CJ4 engine tests, CF and CF-2 engine tests, L-33 Moisture Corrosion Test, High-Temperature Cyclic Durability Test (ASTM D 5579), 288-hour VE engine oil performance test, L-38 standard lubricant test, Denison P46 Piston Pump Test Stand, Sundstrand Dynamic Corrosion Test Stand, a block-on-ring test apparatus, and any test apparatus required for performing test analysis under Mercon®, Mercon® V, Dexron® III, Dexron® III-H, Caterpillar® TO-4, Allison® C4, JASO, GF-4, GF-5, MIL-E, MIL-L, and Sequences II through VIII.

Other embodiments of the invention include lubricating compositions comprising a base oil at least one calcium phenate, at least one zinc dinonyinapthalene sulfonate, at least one zinc dihydrocarbyl dithiophosphate, wherein the fluid is TO-4 compliant having an FZG scuffing load stage score of 12.

Additional objects and advantages of the disclosure will be set forth in part in the description which follows, and/or can be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

### DESCRIPTION OF THE EMBODIMENTS

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include:

(1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical);

(2) substituted hydrocarbon substituents, that is, substituents containing non-hydrocarbon groups which, in the context of this invention, do not alter the predominantly hydrocarbon substituent (e.g., halo (especially chloro and fluoro), hydroxy, alkoxy, mercapto, alkylmercapto, nitro, nitroso, and sulfoxy);

(3) hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents such as pyridyl, furyl, thienyl, and imidazolyl. In general, no more than two, for example, no more than one, non-hydrocarbon substituent will be present for every ten carbon atoms in the hydrocarbyl group; typically, there will be no non-hydrocarbon substituents in the hydrocarbyl group.

As used herein, the term “percent by weight”, unless expressly stated otherwise, means the percentage the recited component represents to the weight of the entire composition.

The terms “oil-soluble” or “dispersible” used herein do not necessarily indicate that the compounds or additives are soluble, dissolvable, miscible, or capable of being suspended in the oil in all proportions. These do mean, however, that they are, for instance, soluble or stably dispersible in oil to an extent sufficient to exert their intended effect in the environment in which the oil is employed. Moreover, the additional incorporation of other additives may also permit incorporation of higher levels of a particular additive, if desired.

As used herein, the term “CAT TO-4 compliant” indicates that a fluid is capable of passing all of the specification requirements of the Jun. 23, 2005, Caterpillar Inc. TO-4 Transmission And Drivetrain Fluid Requirements specification.

### Lubricant Additives Used to Formulate Tractor Oils

Oil of Lubricating Viscosity

Oils of lubricating viscosity (i.e., base oils) suitable for use in formulating embodiments herein may be selected from any of the synthetic oils, mineral oils, or mixtures thereof. In an aspect, the composition can comprise a combination of a vegetable oil and a synthetic oil as disclosed in U.S. Patent Application No. 2005/0059562, published Mar. 17, 2005. Mineral oils include animal oils and vegetable oils (e.g., castor oil, lard oil) as well as other mineral lubricating oils such as liquid petroleum oils and solvent treated or acid-treated mineral lubricating oils of the paraffinic, naphthenic or mixed paraffinic-naphthenic types. Oils derived from coal or shale are also suitable. Further, oils derived from a gas-to-liquid process are suitable.

Hence, the base oil used which can be used to make the compositions as described herein can be selected from any of the base oils in Groups I-V as specified in the American Petroleum Institute (API) Base Oil Interchangeability Guidelines.

Metal Detergent

Embodiments of the present disclosure may comprise two metal detergents. Detergents generally comprise a polar head with a long hydrophobic tail where the polar head comprises a metal salt of an acidic organic compound. The salts may contain a substantially stoichiometric amount of the metal, in which case they are usually described as normal or neutral salts, and would typically have a total base number or TBN (as measured by ASTM D2896) of from about 0 to less than about 150. Large amounts of a metal base may be included by reacting an excess of a metal compound such as an oxide or hydroxide with an acidic gas such as carbon dioxide. The resulting overbased detergent comprises micelles of neutralized detergent surrounding a core of inorganic metal base (e.g., hydrated carbonates). Such overbased detergents may have a TBN of about 150 or greater, such as from about 150 to about 450 or more.

Detergents that may be used in the present embodiments include oil-soluble neutral and overbased sulfonates, phenates, sulfurized phenates, and salicylates of a metal, particularly metals such as, e.g., sodium, potassium, lithium, calcium, magnesium and zinc.

Zinc sulfonates suitable for use in the present invention include zinc dihydrocarbyl aromatic sulfonates such as zinc dialkylnaphthalene sulfonate. The zinc dialkylnaphthalene sulfonate has a sulfonate group attached to one ring of the naphthalene nucleus and an alkyl group attached to each ring. Each alkyl group can independently contain from about six to about twenty carbon atoms, but it is preferred that they contain from about eight to twelve carbon atoms. The dialkylnaphthalene sulfonate group is attached to the zinc through the sulfonate group. A particularly preferred zinc dialkylnaphthalene sulfonate is zinc dinonylnaphthylene sulfonate available commercially as NA-SUL® ZS from King Industries, Inc. or alternatively blends of zinc sulfonates and zinc carboxylates in a ratio of zinc sulfonate to zinc carboxylate of from about 1:3 to about 3:1 parts by weight. Said blends are available commercially from King Industries, Inc. under the tradename NA-SUL® ZS-HT.

### Phosphorus-Based Wear Preventative

The phosphorus-based wear preventative may comprise a metal dihydrocarbyl dithiophosphate compound, such as but not limited to a zinc dihydrocarbyl dithiophosphate compound. Suitable metal dihydrocarbyl dithiophosphates may comprise dihydrocarbyl dithiophosphate metal salts wherein the metal may be an alkali or alkaline earth metal, or aluminum, lead, tin, molybdenum, manganese, nickel, copper, or zinc. The zinc salts are most commonly used in lubricating oil.

Dihydrocarbyl dithiophosphate metal salts may be prepared in accordance with known techniques by first forming a dihydrocarbyl dithiophosphoric acid (DDPA), usually by reaction of one or more alcohol or a phenol with P₂S₅ and then neutralizing the formed DDPA with a metal compound. For example, a dithiophosphoric acid may be made by reacting mixtures of primary and secondary alcohols. Alternatively, multiple dithiophosphoric acids can be prepared where the hydrocarbyl groups on one are entirely secondary in character and the hydrocarbyl groups on the others are entirely primary in character. To make the metal salt, any basic or neutral metal compound could be used but the oxides, hydroxides and carbonates are most generally employed. Commercial additives frequently contain an excess of metal due to the use of an excess of the basic metal compound in the neutralization reaction.

The zinc dihydrocarbyl dithiophosphates (ZDDP) are oil soluble salts of dihydrocarbyl dithiophosphoric acids and may be represented by the following formula:

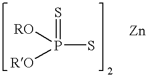

wherein R and R′ may be the same or different hydrocarbyl radicals containing from 1 to 18, for example 2 to 12, carbon atoms and including radicals such as alkyl, alkenyl, aryl, arylalkyl, alkaryl, and cycloaliphatic radicals. R and R′ groups may be alkyl groups of 2 to 8 carbon atoms. Thus, the radicals may, for example, be ethyl, n-propyl, i-propyl, n-butyl, i-butyl, sec-butyl, amyl, n-hexyl, i-hexyl, n-octyl, decyl, dodecyl, octadecyl, 2-ethylhexyl, phenyl, butylphenyl, cyclohexyl, methylcyclopentyl, propenyl, butenyl. In order to obtain oil solubility, the total number of carbon atoms (i.e., R and R′) in the dithiophosphoric acid will generally be about 5 or greater. The zinc dihydrocarbyl dithiophosphate can therefore comprise zinc dialkyl dithiophosphates.

Other suitable components that may be utilized as the phosphorus-based wear preventative include any suitable organophosphorus compound, such as but not limited to, phosphates, thiophosphates, phosphites, and salts thereof and phosphonates. Suitable examples are tricresyl phosphate (TCP), di-alkyl phosphite (like DiButyl Hydrogen Phosphite), and amyl acid phosphate.

Additional Components

In addition to the other components described herein an additive package may comprise, for example, one or more of an ashless dispersant, a rust inhibitor, an antifoam agent, an antioxidant, and a diluent oil. Further optional components may include viscosity modifiers, copper and lead bearing corrosion inhibitors, demulsifying agents, and pour point depressants.

### EXAMPLES

This invention is described in more detail by inventive and comparative examples. The invention should not be limited by these examples; rather they will serve to demonstrate the utility of the invention. The test used to differentiate the inventive FDPT lubricant compositions from commercially available TO-4 lubricants is the FZG Scuffing test method, a wear test, which is described more fully below.

### FZG Scuffing

The FZG scuffing test method is used to evaluate the scuffing load capacity of CAT TO-4 fluids. The test method is ASTM D5182, incorporated herein by reference, which is a standard method used to evaluate the scuffing load capacity of fluids. The test method evaluates gear tooth face scuffing resistance of fluids using “A” profile gears. The rig is operated at 1450 rpm through up to 12 progressive load stages at 15 minute intervals. Standard tests are run at a fluid temperature of 90° C. The test procedure commences with a comparatively small pre-load of the meshed gears and after a 15 minute test duration the gear teeth are inspected for scuffing. If the gear teeth are determined to have a pre-assigned amount of scuffing the test is considered a fail at that load stage and the test is terminated at that point. If the gear teeth do not have a pre-assigned amount of scuffing an additional load is added to the meshed gear teeth and the test run for a further 15 minutes. This mode of operation is continued until either the gear teeth are determined to have failed at a particular load stage or load stage 12 is reached without failure. There are no loads stages above load stage 12, therefore if a fluid is deemed to have acceptable performance after load stage 12 the test is terminated. In addition to a visual evaluation of gear tooth condition, gear weight loss is measured.

In order for a fluid to meet the requirements of CAT TO-4 the performance in the ASTM D5182 FZG scuffing test must meet the minimum required performance standards as follows:

| SAE VISCOSITY GRADE | Minimum Passing Load Stages |
|---|---|
| 10 W | 8 |
| 30 W | 8 |
| 40 W | 10 |
| 50 W | 10 |

### INVENTIVE AND COMPARATIVE EXAMPLES

### Inventive FDPT Fluid

wt. % of an overbased metal phenate and 0.3 wt. % of a metal dialkylnapthalene sulfonate are combined with 1.55 wt. % of a core package containing a rust inhibitor, a pour point depressant, an antioxidant and process oil. This blend is maintained at or below 85° C. and a phosphorous antiwear compound, 1.1 wt. %, is added followed by 0.75 wt. % of an ashless dispersant. The additive blend is mixed for approximately two hours followed by adding a base oil to provide a total additive treat rate of 6.4 wt. %.

Comparative FDPT fluid 1

2.7 wt. % of an overbased metal phenate and 0.3 wt. % of a overbased metal sulfonate are combined with 1.55 wt. % of a core package containing a rust inhibitor, a pour point depressant, an antioxidant and process oil. This blend is maintained at or below 85° C. and a phosphorous antiwear compound, 1.1 wt. %, is added followed by 0.75 wt. % of an ashless dispersant. The additive blend is mixed for approximately two hours followed by adding a base oil to provide a total additive treat rate of 6.4 wt. %.

Comparative FDPT Fluid 2

The comparative FDPT fluid 2 was obtained by purchasing a commercially available CAT TO-4 compliant fluid.

TABLE 1

| | | Comparative | Comparative |
|---|---|---|---|
| | | FDPT 1 | FDPT 2 |
| | Inventive | (Afton fluid | (commercial |
| | FDPT | w/o sulfonate) | fluid) |
| Overbased Metal | 0.0 | 0.3 | N/A |
| Sulfonate | | | |
| Overbased Metal | 2.7 | 2.7 | N/A |
| Phenate | | | |
| metal dialkylnapthalene | 0.3 | 0.0 | N/A |
| sulfonate | | | |
| ZDDP | 1.1 | 1.1 | N/A |
| Core Package | 1.55 | 1.55 | N/A |
| Treat Rate | 6.4 | 6.4 | N/A |
| Base Oil Blend | balance | balance | N/A |
| FDPT Tests | | | |
| CAT TO-4 Compliant | Y | N | Y |
| FZG Scuffing | 12 | 12 | 8 |
| Load Stage | | | |

Explanation of Test Results on Inventive and Comparative Examples

The present invention is a unique combination of additives for use as a heavy-duty transmission fluid. This combination provides high coefficients of friction, excellent oxidation stability, corrosion protection and a high level of anti-wear performance. As shown in Table I above, the inventive composition is CAT TO-4 compliant while also providing an exceptional FZG Scuffing score of 12. Comparative FDPT 1 fluid is not CAT TO-4 compliant because it is unable to provide adequate levels of static and dynamic friction as required by CAT TO-4. Without being bound to any specific theory, it is believed that the absence of zinc sulfonate renders the FDPT 1 fluid deficient in the necessary friction performance to meet the CAT TO-4 specification. Comparative FDPT 2 fluid is a commercially available CAT TO-4 fluid. While the FDPT 2 fluid is CAT TO-4 compliant it significantly underperforms the inventive fluid in terms of wear and corrosion/oxidation performance as evidenced by the lower FZG Scuffing load stage score of 8.

Other embodiments of the present disclosure will be apparent to those skilled in the art from consideration of the specification and practice of the invention disclosed herein. As used throughout the specification and claims, “a” and/or “an” may refer to one or more than one. Unless otherwise indicated, all numbers expressing quantities of ingredients, properties such as molecular weight, percent, weight percent, ratio, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not as an attempt to limit the application by the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the invention being indicated by the following claims.

What is claimed is:

1. A lubricating composition comprising:
a) a base oil;
b) at least one metal phenate;
c) at least one metal dialkylnapthalene sulfonate; and
d) at least one phosphorus-based wear preventative.

2. The composition according to claim 1, wherein said at least one metal phenate is selected from the group consisting of calcium phenate, magnesium phenate, zinc phenate, and mixtures thereof.

3. The composition of claim 1 wherein said metal phenate is calcium phenate.

4. The composition of claim 1 wherein said metal dialkylnapthalene sulfonate is selected from the group consisting of calcium dialkylnapthalene sulfonate, magnesium dialkylnapthalene sulfonate, zinc dialkylnapthalene sulfonate, and mixtures thereof.

5. The composition of claim 4 wherein the metal dialkylnapthalene sulfonate comprises a zinc dinonyinapthalene sulfonate.

6. The composition according to claim 1, wherein the at least one phosphorus-based wear preventative comprises at least one metal dihydrocarbyl dithiophosphate compound.

7. The composition according to claim 6, wherein said at least one metal dihydrocarbyl dithiophosphate compound comprises at least one zinc dihydrocarbyl dithiophosphate compound.

8. The composition according to claim 7, wherein said composition contains from about 200 to about 800 ppm phosphorus from the metal dihydrocarbyl dithiophosphate compound.

9. The composition according to claim 8, wherein said composition contains from about 200 to 400 ppm phosphorus from the metal dihydrocarbyl dithiophosphate compound.

10. An additive package comprising:
a) at least one metal phenate;
b) at least one metal dialkylnapthalene sulfonate; and
c) at least one phosphorus-based wear preventative.

11. The additive package according to claim 10, wherein said at least one metal phenate is selected from the group consisting of calcium phenate, magnesium phenate, zinc phenate, and mixtures thereof.

12. The additive package of claim 11 wherein said metal phenate is calcium phenate.

13. The additive package of claim 12 wherein said metal dialkylnapthalene sulfonate is selected from the group consisting of calcium dialkylnapthalene sulfonate, magnesium dialkylnapthalene sulfonate, zinc dialkylnapthalene sulfonate, and mixtures thereof.

14. The additive package of claim 13 wherein the metal dialkylnapthalene sulfonate comprises a zinc dinonylnapthalene sulfonate.

15. The additive package according to claim 14, wherein the at least one phosphorus-based wear preventative comprises at least one metal dihydrocarbyl dithiophosphate compound.

16. The additive package according to claim 15, wherein said at least one metal dihydrocarbyl dithiophosphate compound comprises at least one zinc dihydrocarbyl dithiophosphate compound.

17. The additive package according to claim 16, wherein said composition contains from about 200 to about 800 ppm phosphorus from the metal dihydrocarbyl dithiophosphate compound.

18. The additive package according to claim 17, wherein said composition contains from about 200 to 400 ppm phosphorus from the metal dihydrocarbyl dithiophosphate compound.

19. The compositions of any one of claims 1-9 wherein the lubricating composition is TO-4 compliant having an FZG scuffing load stage score of 12.

20. A method for improving the FZG scuffing protection of a vehicle engine or transmission comprising the steps of: (1) adding to the vehicle engine or transmission a lubricating oil composition of any one of claims 1-9; and (2) operating the vehicle.

21. A method of lubricating a machine part comprising lubricating said machine part with a lubricant composition of anyone of claims 1-9.

22. The method of claim 21 wherein said machine part comprises a gear, an axle, a differential, an engine, a crankshaft, a transmission, or a clutch.

23. The method of claim 22, wherein said transmission is selected from the group consisting of an automatic transmission, a manual transmission, an automated manual transmission, a semi-automatic transmission, a dual clutch transmission, a continuously variable transmission, and a toroidal transmission.

24. The method of claim 22, wherein said transmission comprises a continuously slipping torque converter clutch, a slipping torque converter, a lock-up torque converter, a starting clutch, one or more shifting clutches, or an electronically controlled converter clutch.

25. The method of claim 22, wherein said gear is selected from the group consisting of an automotive gear, a stationary gearbox, and an axle.

26. The method of claim 22, wherein said gear is selected from the group consisting of a hypoid gear, a spur gear, a helical gear, a bevel gear, a worm gear, a rack and pinion gear, a planetary gear set, and an involute gear.

27. The method of claim 22, wherein said differential is selected from the group consisting of a straight differential, a turning differential, a limited slip differential, a clutch-type limited slip differential, and a locking differential.

28. The method of claim 22, wherein said engine is selected from the group consisting of an internal combustion engine, a diesel engine, a rotary engine, a gas turbine engine, a four-stroke engine, and a two-stroke engine.

29. The method of claim 22, wherein said engine comprises a piston, a bearing, a crankshaft, and/or a camshaft.

30. A method for testing the lubricant properties of a composition using a testing apparatus comprising lubricating said testing apparatus with a lubricant composition as defined in anyone of claims 1-9, said testing apparatus selected from the group consisting of: a Brookfield viscometer, any Vickers Test apparatus, an SAE No. 2 friction test machine, an electric motor-driven Hydra-Matic 4L60-E automatic transmission, ASTM D 471 or D 676 Elastomer Compatibility test equipment, NOACK volatility procedure machine, any test apparatus necessary for ASTM D 2882, D 5182, D 4172, D3233, and D2782 Wear Procedures, ASTM Foaming Procedure apparatus, test apparatus necessary for ASTM D 130 Copper Corrosion test, test equipment specified by the International Harvester Procedure Method BT-9 Rust Control test, test apparatus required by ASTM D 892 Foaming test, test apparatus required by ASTM D 4998 Gear Anti-Wear Performance test, Link M1158 Oil/Friction Machine, L-33-1 Test Apparatus, L-37 Test Apparatus, L-42 Test Apparatus, L-60-1 Test Apparatus, Strama 4-Square Electric Motor-Driven Procedure Machine, FZG Test Apparatus and parts, SSP-180 Procedure Machine, test apparatus for ASTM D 5579 High Temperature Cyclic Durability Procedure, Sauer-Danfoss Series 22 or Series 90 Axial Piston Pump, John Deere Synchro-Plus transmission, an SRV-friction wear tester, a 4-ball test apparatus, an LFW-1 test apparatus, a sprag clutch over-running wear test (SCOWT) apparatus, API CJ-4 engine tests, CF and CF-2 engine tests, L-33 Moisture Corrosion Test, High-Temperature Cyclic Durability Test (ASTM D 5579), 288-hour VE engine oil performance test, L-38 standard lubricant test, Denison P46 Piston Pump Test Stand, Sundstrand Dynamic Corrosion Test Stand, a block-on-ring test apparatus, and any test apparatus required for performing test analysis under Mercon®, Mercon® V, Dexron® III, Dexron® III-H, Caterpillar® TO-4, Allison® C-4, JASO, GF4, GF-5, MIL-E, MIL-L, and Sequences II through VIII.

31. A lubricating composition comprising:
a) a base oil;
b) at least one calcium phenate;
c) at least one zinc dinonylnapthalene sulfonate;
d) at least one zinc dihydrocarbyl dithiophosphate.
e) wherein the lubricating composition is TO-4 compliant having an FZG scuffing load stage score of 12.
